(19) United States
(12) Patent Application Publication
(10) Pub. No.: US 2006/0005834 A1
(43) Pub. Date: Jan. 12, 2006

(54) Method and system of providing therapeutic gas to a patient to prevent breathing airway collapse

(75) Inventors: Alonzo C. Aylsworth, Wildwood, MO (US); Lawrence C. Spector, Austin, TX (US)

Correspondence Address: CONLEY ROSE, P.C., P. O. BOX 3267, HOUSTON, TX, 77253-3267, US

(73) Assignee: ACOBA, LLC, Wildwood, MO (US)

(21) Appl. No.: 10/886,253

(22) Filed: Jul. 7, 2004

(51) Int. Cl. A61M 16/00; A62B 7/00

(52) U.S. Cl. 128/204.21; 128/204.18; 128/204.23

(57) ABSTRACT

A method and system of providing therapeutic gas to a patient to prevent breathing airway collapse during sleep. Some exemplary embodiments may be a method comprising providing a flow of therapeutic gas to a patient during a plurality of inhalations, detecting a flow rate of the therapeutic gas of at least one of the patient's nares during a first inhalation of the plurality of inhalations, and increasing the flow of therapeutic gas in a second inhalation of the plurality of inhalations based on an amount the flow rate of therapeutic gas in the first inhalation is less than a set point therapeutic gas flow.

### CROSS-REFERENCE TO RELATED APPLICATIONS

None.

### STATEMENT REGARDING FEDERALLY SPONSORED RESEARCH OR DEVELOPMENT

Not applicable.

### BACKGROUND OF THE INVENTION

1. Field of the Invention

Embodiments of the present invention are directed to methods and systems of providing breathing airway therapeutic gas flow to treat breathing disorders during sleep. More particularly, embodiments of the invention are directed to methods and systems where the control of therapeutic gas provided to a patient during an inhalation is based on airflow during a previous inhalation and a set point airflow, and is at least partially independently of pressure to achieve the flow.

2. Background of the Invention

Sleep-disordered breathing is common throughout the population and may encompass many conditions, such as snoring, hypopneas and apneas. Apnea may be a disorder where a person temporarily stops breathing during sleep. A hypopnea may be a period of time where a person's breathing becomes abnormally shallow, loosely defined to be a reduction in breathing volume by 50% or more for greater than ten seconds. In some cases, a hypopnea may precede an apnea event. Snoring is a disorder whose cause, in some cases, may be similar to the causes and effects of apnea and hypopnea.

Although snoring, hypopneas and apneas may have multiple causes, one trigger may be full or partial blockages of the patient's breathing airway. In particular, in some patients the pharynx, larynx, upper airway and/or other soft tissue in the respiratory tract may collapse due to forces of gravity, enlarged or swollen airway structures, narrowing and/or forces associated with lower pressure inside the body than outside the body. A collapse of the pharynx, larynx, upper airway and/or other soft tissue in the respiratory tract may thus cause a full or partial blockage, which may lead to snoring, hypopnea and/or apnea events.

Related art methods to counter collapse of the breathing airway may be the application of positive airway pressure, possibly by using a continuous positive airway pressure (CPAP) machine. This may be accomplished in the related art by placing a mask over at least the patient's nose, and providing within the mask a prescribed titration pressure communicated to the breathing airway. The pressure within the breathing airway may be greater than the pressure outside the body, thus holding open or splinting the airway.

In some related art CPAP machines, the doctor-prescribed titration pressure is supplied to the patient continuously regardless of the presence or absence of any breathing abnormality. Other CPAP machines may incorporate an auto-titration feature, which may initially apply a low pressure, and then may increase the pressure after detecting a full or partial collapse of the breathing airway. More particularly, related art devices may observe a patient's inhalation curve which, in the absence of a full or partial collapse, is bell-shaped. By algorithmically determining that the patient's inhalation curve has a flattened peak, the related art devices thus determine that a full or partial collapse of the patient's breathing airway has taken place and increase the applied positive airway pressure. If no flattening of the peak of the bell-shaped curve is detected, yet a second inhalation curve indicates a smaller volume inhaled by the patient, the related art devices either may not change to applied pressure, or reduce pressuring assuming that the patient is having difficulty breathing against the supplied positive airway pressure.

Moreover, related art CPAP devices with the auto-titration feature always tend toward a lower applied positive airway pressure. In other words, if a patient has exhibited no breathing abnormalities over a certain period of time, CPAP devices with the auto-titration feature begin lowering the applied positive airway pressure, e.g., 0.5 centimeters of water every two minutes. The lowering of the applied positive airway pressure continues until a breathing abnormality is detected, and then the positive airway pressure is again raised.

As can be appreciated from the above discussion, related art CPAP devices with the auto-titration feature may intentionally induce breathing abnormalities in a patient as part of the algorithmic mechanism to determine a positive airway pressure where breathing is free of abnormalities. However, patients use CPAP devices in an attempt to alleviate breathing abnormalities, and in this sense CPAP devices with the auto-titration feature fail in their intended purpose. CPAP devices without the auto-titration feature have no means to respond to changes in nasal airway resistance.

Thus, what is needed in the art is a method and related system of addressing sleep-disordered breathing that overcomes the deficiencies of the related art.

### SUMMARY OF SOME OF THE PREFERRED EMBODIMENTS

The problems noted above are solved in large part by a method and system of providing therapeutic gas to a patient to prevent breathing airway collapse during sleep. Some exemplary embodiments may be a method comprising providing a flow of therapeutic gas to a patient during a plurality of inhalations (the flow of therapeutic gas preventing collapse of the patient's breathing airway while the patient sleeps), detecting a flow rate of the therapeutic gas of at least one of the patient's nares during a first inhalation of the plurality of inhalations, and increasing the flow of therapeutic gas in a second inhalation of the plurality of inhalations based on an amount the flow rate of therapeutic gas in the first inhalation is less than a set point therapeutic gas flow (the increasing before the occurrence of a partial or full airway collapse).

Other exemplary embodiments may be a system comprising a blower, a flow sensor fluidly coupled to the blower (the flow sensor measuring therapeutic gas flow provided by the blower, wherein the blower and sensor are fluidly couple to at least one naris of a patient, and wherein the therapeutic gas flow prevents collapse of the patient's breathing airway while the patient sleeps), and a processor electrically coupled to the blower and flow sensor (the processor executing a program that controls the therapeutic gas flow from the blower provided to the patient). The processor, executing a program, reads therapeutic gas flow measured by the flow sensor during a first inhalation of the patient, and the program increases the speed of the blower in a second inhalation, the increase based on an amount the therapeutic gas flow in the first inhalation is less than a set point therapeutic gas flow (and the increasing before the occurrence of a partial or a full airway collapse).

Yet further exemplary embodiments may be a method comprising providing a flow of therapeutic gas to a patient during a plurality of inhalations (the flow of therapeutic gas preventing collapse of the patient's breathing airway while the patient sleeps), detecting a flow rate of therapeutic gas of at least one of the patient's nares during a first inhalation of the plurality of inhalations, and decreasing the flow of therapeutic gas in a second inhalation of the plurality of inhalations based on an amount the flow rate of therapeutic gas in the first inhalation is greater than a set point therapeutic gas flow.

Further exemplary embodiments may be a system comprising a blower, a flow sensor fluidly coupled to the blower (the flow sensor measuring therapeutic gas flow provided by the blower, the blower and flow sensor fluidly couple to at least one naris of the patient, and wherein the therapeutic gas flow prevents collapse of the patient's breathing airway), and a processor electrically coupled to the blower and the flow sensor (the processor executing a program that controls the therapeutic gas flow from the blower provided to the patient). The processor, executing a program, reads therapeutic gas flow measured by the flow sensor during a first inhalation of the patient and decreases the speed of the blower in a second inhalation (the decrease based on an amount of the therapeutic gas flow in the first inhalation is above a set point therapeutic gas flow).

Yet still other embodiments may be a method comprising operating a blower coupled to a motor providing a flow of air at pressures above atmospheric to at least one naris of a patient during a plurality of inhalations of a sleep state of a patient (the flow of air prevents partial or full breathing airway collapse), measuring an airflow through the at least one naris using an airflow detector (the measuring during a first inhalation of the plurality of inhalations), and increasing the blower speed in a second inhalation of the plurality of inhalations based on an amount the airflow in the first inhalation is less than a set point airflow (the increasing before the occurrence of a partial or full airway collapse).

Yet other exemplary embodiments may be a computer-readable medium containing a program that when executed performs a method comprising commanding a blower coupled to a motor to provide a flow of air at pressures above atmospheric to at least one naris of a patient during a plurality of inhalations of a sleep state of the patient (the flow of air prevents partial or fill breathing airway collapse), reading an airflow through the at least one naris using an airflow detector (the reading during a first inhalation of the plurality of inhalations), and commanding an increase in the blower speed in a second inhalation of the plurality of inhalations based on an amount the airflow in the first inhalation is less than a set point airflow (the commanding an increase before the occurrence of a partial or full airway collapse).

Yet further exemplary embodiments may be a method comprising operating a blower coupled to a motor to provide a flow of air at pressures above atmospheric to at least one naris of a patient during a plurality of inhalations (the flow of air prevents partial or full breathing airway collapse during sleep of the patient), measuring an airflow through the at least one naris using an airflow detector, the measuring during a first inhalation of the plurality of inhalations, and decreasing the blower speed in a second inhalation of the plurality of inhalations based on an amount the measured airflow in the second inhalation is above a set point airflow.

The disclosed devices and methods comprise a combination of features and advantages which enable them to overcome the deficiencies of the prior art devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the preferred embodiments of the invention, reference will now be made to the accompanying drawings in which:

FIG. 1 shows an exemplary system for explanation of the relationship of pressure, flow and resistance to flow;

FIG. 2 shows an airway flow control device in accordance with at least some embodiments of the invention;

FIG. 3 shows the relationship between therapeutic gas flow provided to a patient and pressure of the therapeutic gas; and

FIG. 4 shows a flow diagram in accordance with embodiments of the invention.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct connection. Thus, if a first device couples to a second device, that connection may be through a direct connection, or through an indirect connection via other devices and connections.

### DETAILED DESCRIPTION OF THE PREFERRED EMBODIMENTS

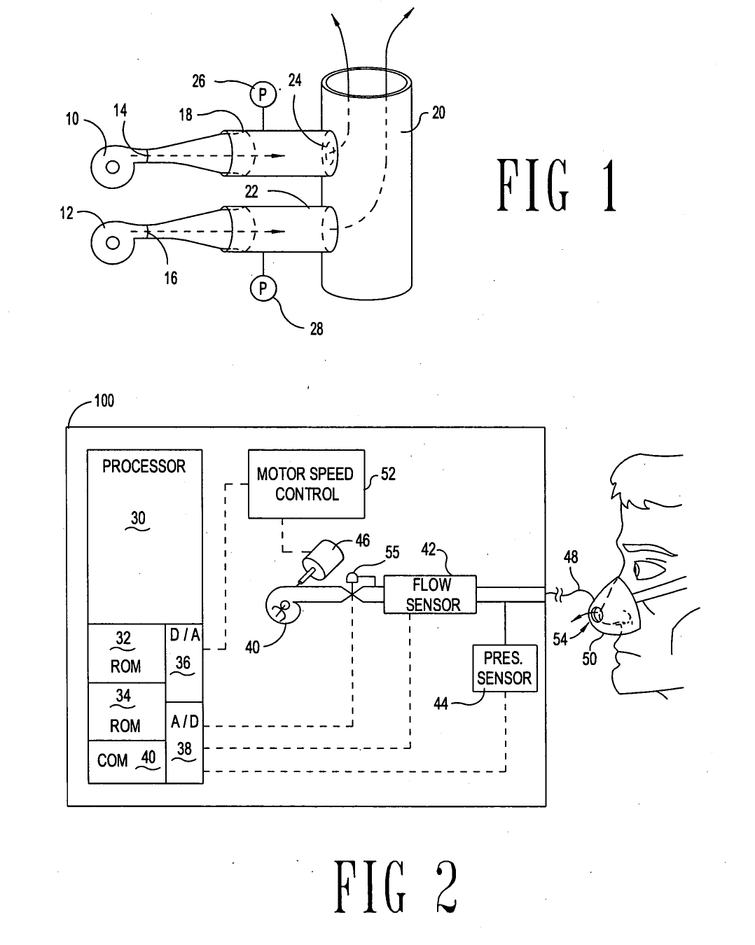

Consider, for purposes of explanation of the relationship between pressure and air flow, the system illustrated in FIG. 1. FIG. 1 illustrates a first fan or blower 10 and a second fan or blower 12. The blowers 10, 12 may be capable of providing controllable flows and/or controllable pressures on their outlet ports 14, 16 respectively. Blower 10 may have its outlet port 14 fluidly coupled to tube 18, and tube 18 may be fluidly coupled to a common chamber 20. Likewise, blower 12 may be fluidly coupled to a tube 22, and the tube 22 may likewise be coupled to the common chamber 20. In the illustration of FIG. 1, tube 18 may fluidly couple to chamber 20 through an orifice 24, and tube 22 may fluidly couple to the common chamber 20 without an orifice (or an orifice having a flow path significantly larger than that of orifice 24). Thus, while each tube 18 and 22 may be fluidly coupled to the common chamber 20, there is a restriction or resistance to air flow from the tube 18 into the chamber 20 by virtue of the orifice 24.

Further consider that each tube 18, 22 has coupled thereto a pressure transducer 26, 28 respectively, each pressure transducers may be capable of reading a pressure within their respective tube 18, 22. Blowers 10, 12 may be operated in a pressure control mode. While the controlled pressure within each tube may be different, for purposes of explanation consider that the pressure within each tube 18, 22 are controlled to be the same. Further consider that the common chamber 20 is at a low pressure, such as vented to atmosphere. Thus, because of the pressure differentials between the tubes 18, 22 and the common chamber 20, there may be an airflow from-the tubes 18, 22 into the chamber 20 (as indicated by the arrows in FIG. 1). However, in spite of the fact that the pressure within the tubes 18, 22 may be the same in this example, the air flow may be different. That is, orifice 24 may provide a resistance to air flow from the tube 18 into the common chamber 20 that is not experienced by air flow moving through tube 22. In particular, because of the restriction caused by orifice 24, the air flow through tube 18 may be less than the air flow through tube 22. Thus, even for the same pressure within the tubes 18, 22, the air flow through those tubes may be different.

Now consider that the blowers 10, 12 are operated in a flow control mode, with each blower attempting to maintain a pre-determined air flow regardless of required pressure. In order to maintain the desired flow, blower 10 may need to develop a higher pressure to overcome the restriction of orifice 24 than the pressure that may be required of blower 12 for the same airflow. With these principles in mind, the specification now turns to the discoveries of the inventors and the methods and related systems flowing from those discoveries.

The inventors of the present specification have found that a person's nasal and upper airway resistance to air flow may have a significant effect on the proper application and viability of positive airway pressure techniques to treat sleep-disordered breathing. In particular, the inventors of the present specification have found that over the course of a sleep session, a person's nasal resistance may change significantly. For example, while sleeping a person may experience periodic swelling of the tissue within one or both of the nares, and this periodic swelling therefore creates periodic increases and decreases of resistance to airflow through the nose. Moreover, a portion of the population may experience full or partial blockages of one or both nares as a function of sleeping position, stage of sleep, and/or irritation of the airway (e.g., such as caused by allergens or excessive applied pressure). For example, a person lying on their back may have a small resistance to airflow through each naris, but one or both nares may become blocked almost instantaneously when that person sleeps on their side or on their stomach.

The changing nasal resistance experienced by some patients may render related art airway flow control devices and techniques unsuitable for their intended purpose. In particular, for CPAP devices that apply only a single pressure (the prescribed titration pressure) throughout the sleep session, increases an airway resistance to airflow may render the CPAP device ineffective. That is, the prescribed titration pressure may provide inadequate flow to ensure that the patient's breathing airway does not collapse given the higher nasal resistance. As for related art CPAP devices with auto-titration features, as mentioned in the Background section, these devices may increase the applied pressure only after a snoring, apnea or hypopnea event. A snoring, apnea and/or hypopnea event may result in an arousal from sleep of the patient. In the situation where the patient's airway resistance is decreasing, the related art auto-titration devices may apply excessive pressure, making it difficult for the patient to exhale and also causing an arousal from sleep of the patient.

Embodiments of the present invention are directed to methods and systems that proactively control therapeutic gas flow to the patient during inhalation (substantially independent of applied pressure) to minimize the occurrence of sleep-disordered breathing, such as snoring, hypopnea and/or apnea events in the patient.

FIG. 2 illustrates an airway flow control device 100 in accordance with at least some embodiments of the invention. The airway flow control device 100 comprises both electrical components and mechanical components. In order to differentiate between electrical connections and mechanical connections, FIG. 2 illustrates electrical connections between components with dashed lines, and fluid connections (e.g., tubing connections between devices) with solid lines. The airway flow control device 100 in accordance with at least some embodiments of the invention comprises a processor 30. The processor 30 may be a microcontroller, and therefore the microcontroller may be integral with read-only memory (ROM) 32, random access memory (RAM) 34, a digital-to-analog converter (D/A) 36, and an analog-to-digital converter (AM) 38. The processor 30 may further comprise a communications logic 40, which allows the airway flow control device 100 to communicate with external devices, e.g., to transfer stored data about the patient's breathing patterns. Although a microcontroller may be preferred because of the integrated components, in alternative embodiments the processor 30 may be implemented by a standalone central processing unit in combination with individual RAM, ROM, communications, D/A and A/D devices.

The ROM 32 may store instructions executable by the processor 30. In particular, the ROM 32 may comprise a software program that implements the various embodiments of flow control for an airway flow control device. The RAM 34 may be the working memory for the processor 30, where data may be temporarily stored and from which instructions may be executed. Processor 30 may couple to other devices within the system by way of the A/D converter 38 and the D/A converter 36.

The airway flow control device in accordance with embodiments of the invention also comprises a fan or blower 40 fluidly coupled to a flow sensor 42 and pressure sensor 44. Blower 40 may be any suitable device, such as a vane-type blower, coupled to an electric motor 46. In alternative embodiments, a source of therapeutic gas, e.g., oxygen, may be used in addition to or in combination with the blower 40. Therapeutic gas pressure and flow created by the blower 40 may thus flow through a flow sensor 42 (of any suitable type) and to a patient's nostrils and/or mouth, possibly through tube 48 and mask 50. In accordance with embodiments of the invention, the airway flow control device 100 provides (substantially independent of applied pressure) a flow of therapeutic gas during inhalation to the patient to minimize sleep-disordered breathing such as snoring, hypopnea and/or apnea events.

As will be more thoroughly discussed below, the primary control parameter for delivery of therapeutic gas in any one inhalation is the flow of therapeutic gas to the patient during a previous inhalation. Control of the flow of therapeutic gas delivered by the airway flow control device may take many forms. In some embodiments, the flow may be controlled by selectively controlling blower 40 speed. For example a motor 46, controlled by a motor speed control circuit 52, may control blower 40. In some embodiments, the motor 46 may be a direct current (DC motor), and therefore motor speed control by the motor speed control circuit 52 may be accomplished by providing a varying voltage DC power to the motor 46. In alternative embodiments, the peak voltage provided to the motor 46 by the motor speed control circuit 52 may remain constant but may be modulated, such as by a pulse width modulation system. In yet other embodiments of the invention, the motor 46 may be an alternating current (AC) motor, and in these embodiments the motor speed control circuit 52 may provide power having varying frequency to the motor 46 to control motor and therefore blower speed. In yet still other embodiments of the invention, the motor 46 may be a stepper motor, and in these embodiments the motor speed control circuit 52 may control the speed the field rotates around the stator to control output shaft speed.

The airway flow control device 100 illustrates that the processor 30 may electrically couple to the motor speed control circuit 52 by way of an analog signal of the D/A converter 36. While communication between the processor 30 and the motor control speed circuit 52 in this manner may be preferred, any communication system that allows the processor to communicate a desired motor speed to the motor speed control circuit 52 would be operable, such as a predetermined plurality of motor speeds selected by delivery of a digital signal between the processor 30 and motor speed control circuit 52, and/or a serial or parallel packet-based communications system in which the processor 30 delivers messages containing the desired motor speed to the motor speed control circuit 52.

In alternative embodiments, the flow of therapeutic gas may be controlled by running the blower 40 at a relatively constant speed, and controlling the flow by control valve 55 at the direction of the processor 30. In yet other embodiments, a combination of controlling the blower 40 speed and the control valve 55 may be utilized.

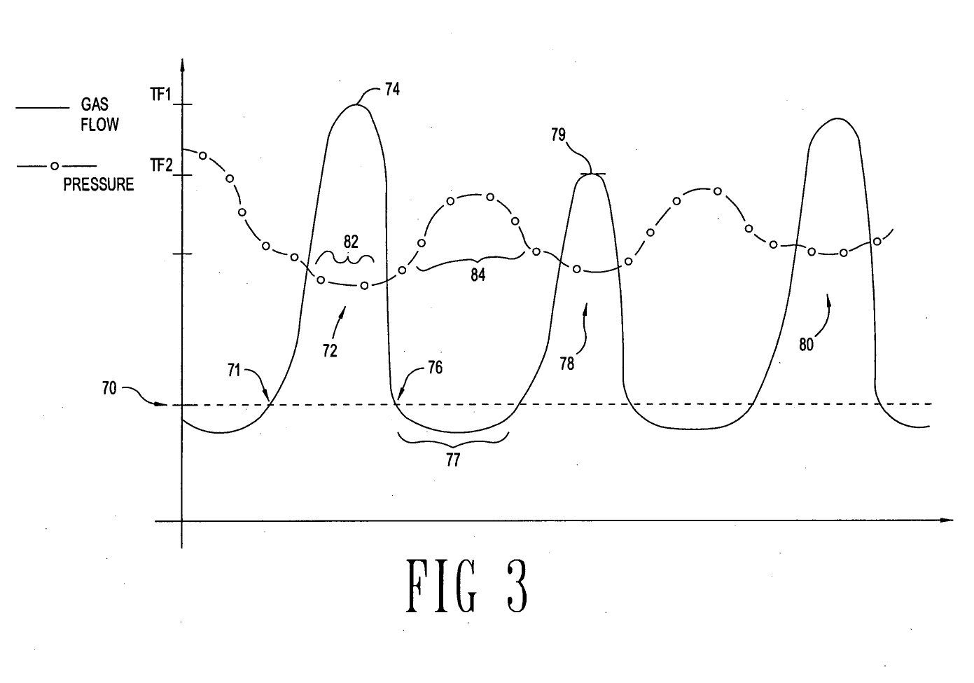

FIG. 3 illustrates the relationship between therapeutic gas flow provided to a patient (possibly as measured by the flow sensor 42) and pressure of the therapeutic gas provided, such as measured by pressure sensor 44. The gas flow, possibly in liters per minute (illustrated by the solid line) shows a bias even at times when the patient is not inhaling. In particular, point 70 illustrates a therapeutic gas flow through the vent port 54 of the mask 50 (see FIG. 2). While at least some therapeutic gas provided by the airway flow control device 100 escapes through vent port 54 at all times, the purpose of the vent port may be to allow escape of gas exhaled by the patient.

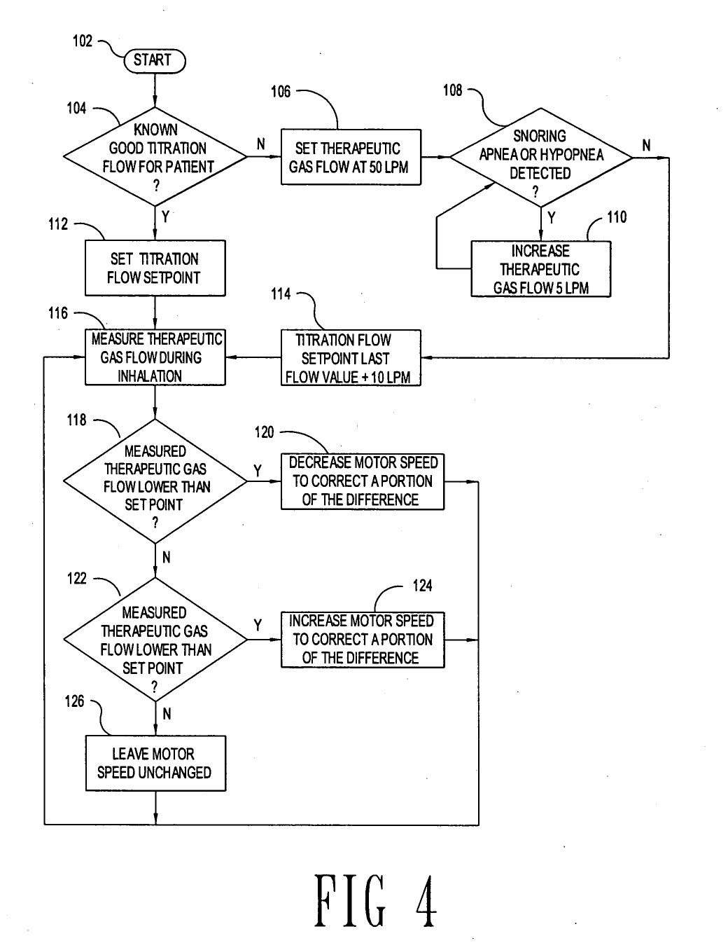

Waveform 72 illustrates a first inhalation of the patient. In particular, the therapeutic gas flow as measure by the flow sensor 42 may initially be the amount that escapes through the vent port 54 (point 71). As the patient inhales the therapeutic gas flow may reach a peak 74, and then trail off again to an amount of flow escaping through the vent port 54 (point 76). As the patient exhales, pressure within mask 50 may increase, and thus therapeutic gas flow may drop as exhaled gases displace therapeutic gas exiting the vent port (as illustrated by portion 77). The peak flow rate of therapeutic gas measured during inhalation (taking into account the flow of approximately 40 liters per minute escaping through the vent port 54) may be on the order of 75 liters per minute; however, this only exemplary and indeed will change from patient to patient. FIG. 3 also illustrates two additional inhalation waveforms 78 and 80, and response of an airway flow control device to the peak flow rates of these waveforms is discussed in relation to the flow diagram of FIG. 4.

Still referring to FIG. 3, the therapeutic gas pressure, possibly as measured by pressure sensor 44, is illustrated in a dash-dot-dash line. In particular, during each inhalation, therapeutic gas inhaled by the patient may result in a reduced gas pressure provided to the patient, as illustrated by portion 82. An airway flow control device 100 in accordance with embodiments of the invention does not attempt to maintain any particular therapeutic gas pressure, and therefore the drop in gas pressure caused by the increased flow of the inhalation preferably does not invoke a control correction on the part of the software executed by the processor 30. As the patient exhales, increased pressure in mask 50 reduces the amount of gas through the flow sensor 42. This in turn results in an increase of pressure as measured by the pressure sensor 44, and as illustrated by portion 84 of the pressure curve.

FIG. 4 illustrates a flow diagram of a software program that may be implemented by the processor 30 in accordance with embodiments of the invention. In particular, the process may start (block 102) and move to a determination of whether there is a known good titration flow for the patient (block 104). If the patient has already used an airway flow control device in accordance with embodiments of the invention, this titration flow may be known and thus there would be no need to perform an auto-titration flow detection mechanism. Alternatively, in the future sleep labs may prescribe a titration flow, and thus the prescribed titration flow could be used directly. Titration flow in accordance with embodiments of the invention shall mean a therapeutic gas flow (in some embodiments air) provided to the patient during an inhalation that is sufficient to prevent at least some sleep-disordered breathing, e.g. as snoring, hypopnea and/or apnea events. The titration flow in accordance with embodiments of the invention is without regard to the pressure required to achieve the flow.

If a titration flow for the patient is not known, the next step in the process may be setting the therapeutic gas flow of the airway flow control device 100 to be an arbitrary starting point below which sleep-disordered breathing is likely to occur. In particular, some embodiments of the invention may make this initial flow setting to be 50 liters per minute (block 106). Other starting flows may be equivalently used. After the initial gas flow is set, the patient is allowed to sleep and a determination is made as to whether the patient snores or experiences a hypopnea and/or apnea event (block 108). If the patient experiences sleep-disordered breathing, the next step in the process may be to increase the therapeutic gas flow and perform the test again. In accordance with at least some embodiments of the invention, the increase may be 5 liters per minute (block 110) and again the patient is monitored for the presence of snoring, hypopnea and/or apnea events. The process continues (blocks 108 and 110) until such time as the patient sleeps without experiencing sleep-disordered breathing. The amount of time that the airway flow control device 100 monitors the patient for sleep-disordered breathing in this auto-titration flow phase may vary from a mere plurality of breaths to several hours. In accordance with at least some embodiments of the invention, the process determines that sleep-disordered breathing is not present at the current therapeutic gas flow set point if no snoring, hypopnea and/or apnea events occur within minutes of the patient falling to sleep.

Still referring to FIG. 4, if the titration flow for the patient is known, that information is provided (block 112) to the airway flow control device 100, possibly by way of a user interface (not specifically shown). If the airway flow control device 100 uses its auto-titration flow feature to determine a titration flow, the titration flow set point may be the flow value determined in steps 108 and 110 plus an arbitrary value to ensure proper operation. In some embodiments this arbitrary value may be on the order of 10 liters per minute (block 114). At this point the airway flow control device 100 may set a motor speed, possibly using the motor speed control circuit 52 (of FIG. 2). The airway flow control device 100, executing a program exemplified by FIG. 4, may then measure the therapeutic gas flow to the patient during an inhalation (block 116). FIG. 3, and in particular waveform 72, is exemplary of a measured flow as a function of time by flow sensor 42 in accordance with some embodiments of the invention.

Measuring therapeutic gas flow in accordance with embodiments of the invention may take many forms. In accordance with some embodiments of the invention, and to simplify the software program executed in the processor 30, the measured therapeutic gas flow is preferably the peak instantaneous flow rate measured during an inhalation. Referring again to FIG. 3, the peak instantaneous flow rate of the inhalation waveform 72 may be the instantaneous therapeutic gas flow rate at point 74. While the inventors have found that using this peak flow rate as the measured therapeutic gas flow works sufficiently well in the algorithms for control of gas flow to a patient, in alternative embodiments the total gas volume inspired by the patient during inhalation (taking into consideration the controlled leak through port 54 and possibly other leaks around the mask) may be equivalently used. In yet other embodiments, the measured therapeutic gas flow could be any value calculated, at least in part, using instantaneous therapeutic gas flow rate determined by flow sensor 42, e.g., average instantaneous flow over the inhalation.

The next step in the exemplary method may be a determination of whether the measured therapeutic gas flow is higher than the titration flow set point (block 118). If the measured therapeutic gas flow is higher, the next step in the process may be decreasing motor 46 speed (block 120), thus decreasing the flow of therapeutic gas flow produced by the blower 40. In accordance with at least some embodiments of the invention however, the decrease in motor speed is a speed decrease to correct only a portion of the difference between the measured therapeutic gas flow and the titration flow set point. Some embodiments may attempt to correct approximately ten percent of the difference between the measured therapeutic gas flow and the titration flow set point (whether the correction is an increase of a decrease). Returning to block 118, if the measured therapeutic gas flow is not higher than the titration flow set point, the next step may be a determination of whether the measured therapeutic gas flow is lower than the titration flow set point (block 120). If so, in the exemplary process an increase in motor speed is effectuated to correct at least a portion of the difference between the measured therapeutic gas flow and the titration flow set point (block 124). If the measured therapeutic gas flow is neither higher than the titration flow set point nor lower than the titration flow set point, the motor speed is left unchanged (block 126), and the process returns to measuring the therapeutic gas flow during the next inhalation (block 116). After motor speed corrections are made (blocks 120 or 124), the process steps to measuring the therapeutic gas flow during the next inhalation (block 116).

Referring somewhat simultaneously to FIGS. 3 and 4, consider for purposes of explanation that a patient is sleeping with an airway flow control device 100 coupled to the patient by way of a mask, such as mask 50 (of FIG. 2). Further consider that the titration flow set point for this particular patient was prescribed or determined to be a value TF 1, as illustrated in FIG. 3 by peak 74. Inasmuch as in this exemplary inhalation of waveform 72 the patient's actual inhalation exactly matched the titration set point, no correction to the provided therapeutic gas flow may be required (block 126 of FIG. 4). Still referring to FIG. 3, on the next inhalation, illustrated by waveform 78, the peak inhaled gas flow rate (point 79) is shown to be below the titration flow set point TF1. In this exemplary situation, the motor speed remained unchanged between the inhalation illustrated by waveform 72 and the illustration by waveform 78, and thus the pressure applied as between these two inhalations remained unchanged. However, increased nasal resistance may have acted to reduce the therapeutic gas flow through the patient's airway, but this reduction may not be significant enough (as yet) to result in collapse of the patient's airway. Unlike related art devices which would see the reduced flow as an increased pressure and therefore reduce pressure, an airway flow control device in accordance with embodiments of the invention increases the motor speed (block 124 of FIG. 4) in an attempt to raise the therapeutic gas flow provided in a subsequent inhalation to compensate for the increased nasal resistance.

Still referring somewhat simultaneously to FIGS. 3 and 4, now consider a situation where the titration flow set point is substantially equal to TF2, and that for a first inhalation as illustrated by waveform 78 the patient achieved the titration flow set point. However, in a subsequent inhalation illustrated by waveform 80 the patient's peak therapeutic gas flow rate exceeded the titration flow set point TF2. Stated otherwise, because the titration flow set point (TF2) of a first inhalation (waveform 78) was met, no change is made in the control of the blower for the subsequent inhalation (block 126 of FIG. 4). However, the patient in this exemplary situation may have experienced a drop in nasal resistance, and for the same applied pressure the therapeutic gas flow went up. Unlike related art devices which would make no change to their applied pressure in spite of the increased size of the inhalation curve (unless that increased size was present for an extended period of time (e.g. several minutes)), an airway flow control device in accordance with embodiments of the invention detects increased therapeutic gas flow (block 118 of FIG. 4) and acts to correct at least a portion of the difference between the peak flow and the titration flow set point (block 120 of FIG. 4). Because of the change, on subsequent inhalations (not shown in FIG. 3) the peak gas flow will asymptotically approach the titration flow set point.

The above discussion is meant to be illustrative of the principles and various embodiments of the present invention. Numerous variations and modifications will become apparent to those skilled in the art once the above disclosure is fully appreciated. For example, while the airway flow control device 100 is shown in FIG. 2 coupled to a nasal-only face mask 50, other masks may be used, such as masks that cover the nose and mouth, mouth pieces alone (possibly in combination with sealing the nose), tubing that seals on an outer or inner portion of each naris, and the like. Moreover, while the discussion of the various embodiments indicates that flow is provided without regard to applied pressure, it will be understood that there may be pressure limits, both on the upper and the lower end, beyond which the device may not traverse. For example, for safety reasons an airway flow control device in accordance with embodiments of the invention may not apply a pressure of greater than 20 centimeters of water, as pressures greater than this may over-inflate and/or damage the patient's airway. Thus, while the airway flow control device 100 is illustrated to include a pressure sensor 44, this device is not strictly required, and instead the device may be implemented using an over-pressure switch and/or a pressure relief valve without departing from the scope and spirit of the invention. Further, in the description of the various embodiments it is assumed that motor speed remains constant during exhalation; however, in alternative embodiments motor speed may be reduced during exhalation, and then returned to the desired speed either just prior to inhalation, or as the patient begins to inhale. Finally, while many of the embodiments of the invention are discussed in relation to supplying a flow of therapeutic gas during sleep, an airway flow control device in accordance with embodiments of the invention may be used by a patient at any time. It is intended that the following claims be interpreted to embrace all such variations and modifications.

What is claimed is:

1. A method comprising:
providing a flow of therapeutic gas to a patient during a plurality of inhalations, the flow of therapeutic gas preventing collapse of the patient's breathing airway;
detecting a flow rate of the therapeutic gas of at least one of the patient's nares during a first inhalation of the plurality of inhalations; and
increasing the flow of therapeutic gas in a second inhalation of the plurality of inhalations based on an amount the flow rate of therapeutic gas in the first inhalation is less than a set point therapeutic gas flow, the increasing before the occurrence of a partial or full airway collapse.

2. The method as defined in claim 1 further comprising detecting a peak flow rate of the therapeutic gas during the first inhalation, and increasing the therapeutic gas flow in the second inhalation based on the peak flow rate of the first inhalation.

3. The method as defined in claim 1 further comprising increasing the flow of therapeutic gas to compensate for a portion of the amount the flow rate of therapeutic gas is below the set point therapeutic gas flow rate.

4. The method as defined in claim 3 further comprising increasing the flow rate by approximately ten percent of the amount needed to correct the amount the flow rate of therapeutic gas is below the set point therapeutic gas flow rate.

5. The method as defined in claim 3 further comprising increasing the flow of therapeutic gas in the second inhalation being immediately subsequent to the first inhalation.

6. A system comprising:
a blower;
a flow sensor fluidly coupled to the blower, the flow sensor measuring therapeutic gas flow provided by the blower, wherein the blower and flow sensor fluidly couple to at least one naris of a patient, and wherein the therapeutic gas flow prevents collapse of the patient's breathing airway; and
a processor electrically coupled to the blower and flow sensor, the processor executing a program that controls the therapeutic gas flow from the blower provided to the patient;
wherein the processor, executing a program, reads therapeutic gas flow measured by the flow sensor during a first inhalation of the patient, and wherein the program increases the speed of the blower in a second inhalation in relation to the speed of the blower during the first inhalation, the increase based on an amount the therapeutic gas flow in the first inhalation is less than a set point therapeutic gas flow, and the increasing before the occurrence of a partial or full airway collapse.

7. The system as defined in claim 6 further comprising:
a motor mechanically coupled to the blower providing rotation of the blower; and
a motor speed control circuit electrically coupled to the motor and the processor;
wherein the processor, executed the program, commands the motor speed control circuit to increase the speed of the motor.

8. The system as defined in claim 6 wherein the processor, executing the program, increases the speed of the blower to compensate for a portion of the amount the therapeutic gas flow in the first inhalation is less than the set point therapeutic gas flow.

9. The system as defined in claim 8 wherein the processor, executing the program, increases the speed of the blower approximately ten percent of the amount needed to correct the amount the therapeutic gas flow is less than the set point therapeutic gas flow.

10. A method comprising:
providing a flow of therapeutic gas to a patient during a plurality of inhalations, the flow of therapeutic gas preventing collapse of the patient's breathing airway;
detecting a flow rate of the therapeutic gas of at least one of the patient's nares during a first inhalation of the plurality of inhalations; and
decreasing the flow of therapeutic gas in a second inhalation of the plurality of inhalations based on an amount the flow rate of therapeutic gas in the first inhalation is greater than a set point therapeutic gas flow.

11. The method as defined in claim 10 further comprising detecting a peak flow rate of the therapeutic gas during the first inhalation, and decreasing the flow of therapeutic gas in the second inhalation based on the peak flow rate of the first inhalation.

12. The method as defined in claim 10 further comprising decreasing the flow of therapeutic gas to compensate for a portion of an amount the flow rate of therapeutic gas in the first inhalation is above the set point therapeutic gas flow rate.

13. The method as defined in claim 12 further comprising decreasing the flow of therapeutic gas by approximately ten percent of the amount needed to correct the amount the detected flow rate of therapeutic gas in the first inhalation is above the set point therapeutic gas flow rate.

14. The method as defined in claim 10 further comprising decreasing the flow of therapeutic gas in the second inhalation being immediately subsequent to the first inhalation.

15. A system comprising:
a blower;
a flow sensor fluidly coupled to the blower, the flow sensor measuring therapeutic gas flow provided by the blower, wherein the blower and flow sensor fluidly couple to at least one naris of a patient, and wherein the therapeutic gas flow prevents collapse of the patient's breathing airway; and
a processor electrically coupled to the blower and flow sensor, the processor executing a program that controls the therapeutic gas flow from the blower provided to the patient;
wherein the processor, executing a program, reads therapeutic gas flow measured by the flow sensor during a first inhalation of the patient, and wherein the program decreases the speed of the blower in a second inhalation in relation to the speed of the blower during the first inhalation, the decrease based on an amount the therapeutic gas flow in the first inhalation is above a set point therapeutic gas flow.

16. The system as defined in claim 15 further comprising:
a motor mechanically coupled to the blower providing rotation of the blower, and
a motor speed control circuit electrically coupled to the motor and the processor;
wherein the processor, executed the program, commands the motor speed control circuit to decrease the speed of the motor.

17. The system as defined in claim 15 wherein the processor, executing the program, decreases the blower speed to compensate for a portion of the amount the therapeutic gas flow in the first inhalation is less than the set point therapeutic gas flow.

18. The system as defined in claim 17 wherein the processor, executing the program, decreases the blower speed approximately ten percent of the amount needed to correct the amount the therapeutic gas flow in the first inhalation is less than the set point therapeutic gas flow.

19. A method comprising:
operating a blower coupled to a motor providing a flow of air at pressures above atmospheric to at least one naris of a patient during a plurality of inhalations of a sleep state of the patient, the flow of air prevents partial or full breathing airway collapse;
measuring an air flow through the at least one naris using an air flow detector, the measuring during a first inhalation of the plurality inhalations; and
increasing the blower speed in a second inhalation of the plurality of inhalations based on an amount the air flow in the first inhalation is less than a set point air flow, the increasing before the occurrence of a partial or full airway collapse.

20. The method as defined in claim 19 further comprising measuring a peak air flow rate during the first inhalation, and increasing the air flow in the second inhalation based on the peak air flow rate of the first inhalation.

21. The method as defined in claim 19 further comprising increasing the blower speed to compensate for a portion of the amount the air flow of the first inhalation is below the set point air flow.

22. The method as defined in claim 21 further comprising increasing the blower speed by approximately ten percent of the amount needed to correct the amount the air flow in the first inhalation is below the set point air flow.

23. The method as defined in claim 21 further comprising at least one selected from the group: increasing the voltage applied to the motor; increasing the duty cycle of the voltage waveform applied to the motor; increasing the frequency of the signal applied to the motor; or increasing the speed at which direct current voltage are applied windings of the motor.

24. The method as defined in claim 19 further comprising increasing the blower speed in the second inhalation being immediately subsequent to the first inhalation.

25. A computer readable medium containing a program that when executed performs a method comprising:
commanding a blower coupled to a motor to provide a flow of air at pressures above atmospheric to at least one naris of a patient during a plurality of inhalations of a sleep state of the patient, the flow of air prevents partial or full breathing airway collapse;
reading an air flow through the at least one naris using an air flow detector, the reading during a first inhalation of the plurality inhalations; and
commanding an increase in blower speed in a second inhalation of the plurality of inhalations based on an amount the air flow in the first inhalation is less than a set point air flow, the commanding an increase before the occurrence of a partial or fall airway collapse.

26. The computer readable medium as defined in claim 25 wherein the method further comprises determining a peak air flow rate during the first inhalation.

27. The computer readable medium as defined in claim 25 wherein the method further comprises commanding the increase in blower speed to compensate for a portion of the amount the air flow in the first inhalation is below the set point air flow.

28. The computer readable medium as defined in claim 27 wherein the method further comprises commanding the increase in blower speed to be approximately ten percent of the amount needed to correct the amount the air flow in the first inhalation is below the set point air flow.

29. The computer readable medium as defined in claim 27 wherein the method further comprises at least one selected from the group: commanding an increase in the voltage applied to the motor; commanding an increase in the duty cycle of the voltage waveform applied to the motor, commanding an increase in the frequency of the signal applied to the motor; or commanding an increase in the speed at which direct current voltage are applied windings of the motor.

30. A method comprising:
operating a blower coupled to a motor to provide a flow of air at pressures above atmospheric to at least one naris of a patient during a plurality of inhalations, the flow of air prevents partial or fill breathing airway collapse during sleep of the patient;
measuring an air flow through the at least one naris using an air flow detector, the measuring during a first inhalation of the plurality inhalations; and
decreasing the blower speed in a second inhalation of the plurality of inhalations based on an amount the measured air flow in the second inhalation is above a set point air flow.

31. The method as defined in claim 30 further comprising measuring a peak air flow rate during the first inhalation, and decreasing the air flow in the second inhalation based on the peak air flow rate of the first inhalation.

32. The method as defined in claim 30 further comprising decreasing the blower speed to compensate for a portion of the amount the air flow in the first inhalation is above the set point air flow.

33. The method as defined in claim 32 further comprising decreasing the blower speed by approximately ten percent of the amount needed to correct the amount the air flow in the first inhalation is above the set point air flow.

34. The method as defined in claim 32 further comprising at least one selected from the group: decreasing the voltage applied to the motor; decreasing the duty cycle of the voltage waveform applied to the motor; and decreasing the frequency of the signal applied to the motor.

35. The method as defined in claim 30 further comprising decreasing the blower speed in the second inhalation being immediately subsequent to the first inhalation.
